(19) United States
(12) Patent Application Publication
(10) Pub. No.: US 2008/0076825 A1
(43) Pub. Date: Mar. 27, 2008

(54) Novel Crystalline Forms of Entacapone and Production Thereof

(76) Inventors: Thomas Bader, Zurich (CH); Markus Furegati, Pittsburg, PA (US); Oliver Jungmann, Donaueschingen (DE)

Correspondence Address: HAMMER & HANF, PC, 3125 SPRINGBANK LANE, SUITE G, CHARLOTTE, NC, 28226, US

(21) Appl. No.: 10/584,900

(22) PCT Filed: Dec. 27, 2004
(86) PCT No.: PCT/CH04/00754

§ 371 Date: Apr. 3, 2007

(30) Foreign Application Priority Data
Dec. 31, 2003 (CH) PCT/CH03/00853
Jun. 4, 2004 (CH) 940/04

(51) Int. Cl. A61K 31/277; A61K 31/195; A61P 25/16; C07C 255/42

(52) U.S. Cl. 514/567; 514/619; 558/401

(57) ABSTRACT

The invention relates to three novel forms of entacapone. These are peripheral and selective COMT inhibitors which in combination with levodopa and decarboxylase inhibitors can be used to treating Parkinson's syndrome. Said novel forms arise by crystallization of entacapone in respectively determined conditions. In certain cases, entacapone can be used as a raw product, in a special case it can be used in situ as the product of Knoevenagel condensation of 3,4-dihydroxy-5-nitro-benzaldehyde and N,N-diethyl-2-cyanoacetamide. Also disclosed are improvements of said condensation in relation to the catalyst and the production of the two components thereof.

Entacapone is the short name for (E)-N,N-diethyl-2-cyano-3-(3,4-dihydroxy-5-nitrophenyl)acrylamide. Entacapone is a selective peripheral catechol-O-methyl-transferase (COMT) inhibitor which is used in combination with levodopa (L-dopa) and a decarboxylase inhibitor (e.g. carbidopa) for the treatment of Parkinson's disease. It increases the bioavailability of L-dopa and also prolongs its duration of action. This effect allows a 10-30% reduction of the amount of L-dopa to be administered, by lengthening the dosage interval and/or reducing the single dose of L-dopa. The preparation is marketed under the name COMTAN® or COMTESS®.

The substance “N,N-diethyl-2-cyano-3-(3,4-dihydroxy-5-nitrophenyl)acrylamide” is described and claimed per se in U.S. Pat. No. 5,446,194 and in patents belonging to the same family in European countries, such as DE 37 40 383, GB 2 200 109 and CH 685 426, but said patents do not contain more precise details concerning the configuration or isomer composition of this compound.

According to the patents cited above, N,N-diethyl-2-cyano-3-(3,4-dihydroxy-5-nitrophenyl)acrylamide is synthesized by a Knoevenagel condensation of 3,4-di-hydroxy-5-nitrobenzaldehyde (obtained by the demethylation of 5-nitrovanillin with HBr) and N,N-diethyl-2-cyanoacetamide. This Knoevenagel condensation is carried out in the presence of a catalytic amount of piperidine/acetic acid as catalyst. As already mentioned, however, these patents neither contain details concerning the composition of the product mixture (E/Z isomers), nor describe methods of separating this mixture and purifying its components (yield: 73%). Only patents EP 0 426 468 and U.S. Pat. No. 5,135,950 (cf. below) indicate the composition of the crude product obtained from the Knoevenagel condensation (70-80% of E isomer and 30-20% of Z isomer).

Patents EP 0 426 468 and U.S. Pat. No. 5,135,950, just cited, protect the polymorphous form A of entacapone, i.e. (E)-N,N-diethyl-2-cyano-3-(3,4-dihydroxy-5-nitro-phenyl)acrylamide, and its preparation. As well as the polymorphous form A, said patents also mention another polymorphous form, B, but do not list any data for this polymorphous form B. The claims refer solely to the crystallographically substantially pure polymorphous form A and its preparation. According to the information in said patents, “crystallographically substantially pure polymorphous form A of entacapone” means that at most 3% and preferably at most 2% of another polymorphous form or the Z isomer is present. To prepare entacapone in the crystallographically substantially pure polymorphous form A according to said patents, the crude product obtained from the Knoevenagel condensation (70-80% of E isomer and 30-20% of Z isomer) is dissolved in acetic acid, treated with catalytic amounts of HBr or HCl and then heated to 90° C. On slow cooling, the product crystallizes out in the desired polymorphous form A (yield: 80%).

Within the framework of the present invention, it has now been found that entacapone can be converted to three novel polymorphous forms, hereafter designated as “form C”, “form D” and “form E”.

Form C of entacapone is characterized by the following XRD data:

TABLE 1
XRD data for the polymorphous form C of entacapone

| | Lattice spacing | Rel. intensity |
|---|---|---|
| Angle 2 theta (°) | d (Å) | I/Imax (%) |
| 5.61 | 15.77 | 100 |
| 11.43 | 7.78 | 1 |
| 14.75 | 6.06 | 2 |
| 17.23 | 5.21 | 5 |
| 18.81 | 4.78 | 2 |
| 20.89 | 4.32 | 1 |
| 23.13 | 3.92 | 17 |
| 25.23 | 3.62 | 2 |
| 26.87 | 3.41 | 3 |
| 29.03 | 3.18 | 1 |
| 32.17 | 2.90 | 2 |

Note:
The intensities may vary in known manner due to texture effects.

Form D of entacapone is characterized by the following XRD data:

TABLE 2
XRD data for the polymorphous form D of entacapone

| | Lattice spacing | Rel. intensity |
|---|---|---|
| Angle 2 theta (°) | d (Å) | I/Imax (%) |
| 6.84 | 12.95 | 99 |
| 11.84 | 7.51 | 6 |
| 12.12 | 7.34 | 7 |
| 13.52 | 6.59 | 49 |
| 14.8 | 6.04 | 23 |
| 15.56 | 5.75 | 40 |
| 16.54 | 5.42 | 31 |
| 16.9 | 5.30 | 22 |
| 17.98 | 4.99 | 37 |
| 18.84 | 4.77 | 12 |
| 19.06 | 4.72 | 13 |
| 20.72 | 4.36 | 18 |
| 21.44 | 4.22 | 28 |
| 22.24 | 4.07 | 12 |
| 23.4 | 3.88 | 22 |
| 24 | 3.79 | 39 |
| 24.62 | 3.70 | 76 |
| 25.34 | 3.60 | 51 |
| 26.5 | 3.46 | 65 |
| 27.44 | 3.35 | 100 |
| 28.08 | 3.28 | 51 |
| 29.24 | 3.16 | 15 |
| 29.98 | 3.09 | 17 |

Note:
The intensities may vary in known manner due to texture effects.

Form E of entacapone is characterized by the following XRD data:

TABLE 3
XRD data for the polymorphous form E of entacapone

| | Lattice spacing | Rel. intensity |
|---|---|---|
| Angle 2 theta (°) | d (Å) | I/Imax (%) |
| 6.62 | 13.35 | 100 |
| 8.87 | 9.97 | 4 |
| 12.36 | 7.16 | 8 |
| 12.90 | 6.86 | 12 |
| 13.38 | 6.62 | 11 |
| 14.40 | 6.15 | 5 |
| 15.52 | 5.71 | 49 |
| 17.92 | 4.95 | 33 |
| 18.25 | 4.86 | 22 |
| 19.20 | 4.62 | 6 |
| 20.48 | 4.24 | 26 |
| 21.10 | 4.21 | 7 |
| 21.85 | 4.07 | 6 |
| 22.45 | 3.96 | 6 |
| 22.90 | 3.88 | 7 |
| 24.00 | 3.71 | 30 |
| 24.64 | 3.61 | 36 |
| 25.85 | 3.45 | 77 |
| 27.32 | 3.26 | 20 |

Note:
The intensities may vary due to texture effects.

The crystalline forms C, D and E of entacapone according to the invention are suitable for use as therapeutic active ingredients. Using common auxiliary substances, they can be processed by generally conventional methods to drugs which contain the crystalline form C and/or the crystalline form D and/or the crystalline form E of entacapone and a therapeutically inert excipient. Advantageously, these drugs additionally contain levodopa and a decarboxylase inhibitor, e.g. carbidopa.

Because different crystalline forms of a pharmaceutical active ingredient normally have different bioavailabilities, solubilities and dissolution rates, the novel crystalline forms C, D and E of entacapone broaden the possibilities for the medicinal treatment of patients. Thus it can be of great benefit to the patient if e.g. the bioavailability of commercially available entacapone (which is only 35%) is increased by virtue of the properties of these novel crystalline forms, thereby enabling the dose to be reduced or the dosage intervals lengthened. This would not only reduce the unwanted side effects of entacapone, which in particular occur more frequently at higher dosage than at lower dosage, but also reduce the costs of medication.

According to the invention, the crystalline forms C and/or D and/or E of entacapone, optionally in combination with levodopa and a decarboxylase inhibitor such as carbidopa, can be used for the treatment of Parkinson's disease or for the preparation of corresponding drugs.

The crystalline form C of entacapone can be prepared according to the invention by crystallizing entacapone from a mixture of at least one aromatic and at least one aliphatic hydrocarbon, the aromatic hydrocarbon used preferably being toluene and the aliphatic hydrocarbon used preferably being n-heptane. Other aromatic and aliphatic hydrocarbons suitable for this purpose are benzene and alkyl-substituted derivatives, e.g. p-xylene, o-xylene, ethylbenzene and the like, and n-pentane, n-hexane, petroleum ether and the like. The temperature naturally depends to a certain extent on the hydrocarbons used; advantageously, it generally ranges from about room temperature to about 100° C.

An example of a possible procedure is to dissolve entacapone in toluene, with heating, and add the solution to n-heptane heated to about 95° C., causing immediate crystallization. Crude or purified entacapone can be used here. The crystallized solid of the form C of entacapone can be obtained by filtration at about 90° C. or by filtration after cooling to about room temperature and standing for several hours, e.g. about 14 hours.

The crystalline form D of entacapone can be prepared according to the invention by
a) dissolving entacapone in a water-miscible solvent and adding this solution to water or a mixed aqueous system, causing immediate crystallization; or
b) crystallizing entacapone from a non-acidic solvent or a solvent mixture with at least one non-acidic component, in the presence of a strong acid.

For process variant a) it is possible to use crude or purified entacapone, but not an E/Z mixture such as that obtained in the Knoevenagel condensation of 3,4-dihydroxy-5-nitrobenzaldehyde and N,N-diethyl-2-cyanoacetamide.

For process variant b), on the other hand, it is perfectly possible to use the product of this Knoevenagel condensation of 3,4-dihydroxy-5-nitro-benzaldehyde and N,N-diethyl-2-cyanoacetamide in situ, as an E/Z isomer mixture, without it being necessary to isolate it first and separate it into its constituents; on treatment with a strong acid, the Z isomer, which makes up about 30% of the isomer mixture, is largely converted to the E isomer, the latter being obtained predominantly in the polymorphous form D.

The strong acid used for process variant b) is preferably hydrogen bromide; other acids suitable for this purpose are hydrogen chloride, hydrogen iodide, sulfuric acid in the presence of alkali metal halides, and the like.

Advantageously, the crystallization to form D takes place according to process variant a) in a mixture of water and at least one water-miscible organic solvent, preferably THF/water, acetone/water, acetone/DMSO/water or n-propanol/water, and according to process variant b) by acid treatment in a non-acidic solvent or a mixture of organic solvents with at least one non-acidic component, preferably toluene/acetonitrile or toluene/acetonitrile/acetic acid.

Process variant b) is preferred within the framework of the present invention, and in a particularly preferred embodiment of this process variant b) the strong acid used is hydrogen bromide and the solvent mixture used is toluene/acetonitrile/acetic acid.

The temperature naturally depends to a certain extent on the process variant and reaction medium used; advantageously, it generally ranges from about −10° C. to about 30° C., but for the isopropanol/hexane system mentioned in connection with process variant b) it ranges from about 0° C. to about 68° C.

The crystalline form E of entacapone can be prepared according to the invention by dissolving entacapone in a polar aprotic or alcoholic solvent and adding this solution to an aliphatic hydrocarbon immiscible with this solvent, in which entacapone is insoluble.

Advantageously, the crystallization to form E takes place in a mixture of a polar aprotic or alcoholic organic solvent and an aliphatic hydrocarbon immiscible therewith, preferably THF/n-hexane, THF/n-pentane, THF/cyclohexane or isopropanol/n-hexane.

Form E of entacapone can be prepared using crude or purified entacapone, but not an E/Z mixture such as that obtained in the Knoevenagel condensation of 3,4-dihydroxy-5-nitrobenzaldehyde and N,N-diethyl-2-cyanoacetamide.

As mentioned earlier, the starting material used to prepare the polymorphous form D of entacapone according to process variant b) can be the product of a Knoevenagel condensation of 3,4-dihydroxy-5-nitro-benzaldehyde and N,N-diethyl-2-cyanoacetamide in situ. This Knoevenagel condensation can be improved according to the invention by using diethylamine/acetic acid as catalyst. Compared with the piperidine/acetic acid catalyst used in the condensation according to the state of the art, this has the advantage that an impurity which is very difficult to remove, namely the entacapone analog with a piperidino group in place of the diethylamino group, cannot form.

This Knoevenagel condensation can also be improved according to the invention by preparing the N,N-diethyl-2-cyanoacetamide used by reacting cyanoacetic acid with diethylamine in the presence of dicyclohexylcarbodiimide. Compared with the conventional process for the preparation of N,N-diethyl-2-cyanoacetamide, this is advantageous inasmuch as it avoids low yields or the use of relatively expensive chemicals (such as 2-chloro-N,N-diethylacetamide or butyllithium) or conditions that cannot easily be produced on the industrial scale (such as −70° C.), and inasmuch as the product can be used in the Knoevenagel condensation without prior purification, which considerably simplifies the process by reducing the energy requirement (no need for a high-vacuum distillation) and the waste products, and delivers high yields.

Finally, the Knoevenagel condensation can be improved according to the invention by preparing the 3,4-dihydroxy-5-nitrobenzaldehyde used by the demethylation of 5-nitrovanillin with AlCl₃/pyridine in chlorobenzene. The resulting 3,4-dihydroxy-5-nitrobenzaldehyde is obtained in high yield and its good purity enables it to be used in the Knoevenagel condensation as the crude product without prior purification.

The Knoevenagel condensation can advantageously be carried out by heating 3,4-dihydroxy-5-nitrobenzaldehyde, crude N,N-diethyl-2-cyanoacetamide, acetic acid and diethylamine in toluene, the water formed being removed by azeotropic distillation using a water separator.

After the Knoevenagel reaction (E/Z isomer ratio=70/30), the procedure can advantageously be as follows: Acetonitrile is added to the reaction mixture to dissolve oily constituents. The resulting solution is treated with active charcoal and then, while still hot, added dropwise to cold toluene, after which a 33% solution of HBr in acetic acid is added. The isomerization takes place slowly, the proportion of the E isomer increasing to ≧90%. The suspension obtained is then filtered and the crude product is elutriated in a mixture of 2-propanol and water. For further purification the crude product is dissolved in acetone/water (10/1), with heating, and the solution is added dropwise to an ice-cold mixture of acetone and water (5/95). After elutriation of the moist product in water, entacapone is obtained in a uniform polymorphous form with an HPLC purity of 99.7%.

A study of the polymorphous forms during the process showed that the novel polymorphous form D, possibly partially mixed with the novel form C and/or the novel form E, is already present after the first precipitation from cold toluene. In addition, the form D can be converted to a mixture of the forms C and D in the elutriation from 2-propanol/water. The almost pure polymorphous form D is obtained in the last precipitation of entacapone from acetone and water. The overall yield is >70%.

The Examples which follow are intended to illustrate the invention in greater detail, but without in any way limiting its scope.

### EXAMPLE 1

### Preparation of 3,4-dihydroxy-5-nitrobenzaldehyde

175.0 g of 5-nitrovanillin and 135.1 g of aluminum chloride were suspended in 774.2 g of chlorobenzene to form an orange-red suspension. 319.9 g of pyridine were then added dropwise in such a way that the internal temperature did not exceed 25° C. The deep red suspension obtained after the addition was heated to an internal temperature of 70-80° C. When the reaction was complete, a solution of 525 g of water and 603.75 g of 32% hydrochloric acid (semiconcentrated hydrochloric acid) was added slowly to the reaction mixture. The hydrolysis initially produced a deep red two-phase mixture, from which a yellow solid precipitated out towards the end. When the addition of the semi-concentrated hydrochloric acid had ended, the suspension was concentrated to half the volume under vacuum. 475 g of water were then added to the suspension and the mixture was heated to the boil, during which the solid dissolved. After 5-10 min under reflux, the solution was left to cool slowly and a solid then precipitated out. The suspension was cooled to 20-25° C., stirred at this temperature and then filtered with suction. The solid was washed with 1000 g of water and dried at 60° C. under vacuum (yield: 152.47 g).

### EXAMPLE 2

### Preparation of N,N-diethyl-2-cyanoacetamide

25.0 g of cyanoacetic acid were dissolved in 163.08 g of ethyl acetate. 21.70 g of diethylamine were added slowly to the resulting colorless solution in such a way that the internal temperature did not exceed 25° C. A solution of 61.10 g of dicyclohexylcarbodiimide in 54.06 g of ethyl acetate was then added dropwise and a solid precipitated out slowly. After the addition the suspension was stirred overnight at 35-40° C. When the reaction had ended, the suspension was cooled to 20-25° C. and filtered with suction. The solid was rinsed with 64.87 g of ethyl acetate. The combined filtrates were concentrated under vacuum and a solid precipitated out. The suspension was taken up in 45.05 g of ethyl acetate, the mixture was stirred at 20-25° C. and the solid was filtered off and rinsed with 45.05 g of ethyl acetate. The combined filtrates were concentrated under vacuum again. The residue was taken up in 18.02 g of ethyl acetate, the mixture was filtered, the material on the filter was rinsed with 13.52 g of ethyl acetate and the filtrate was concentrated under vacuum. The crude product obtained was then distilled under vacuum (vapor temperature: 107-110° C., pressure: 4×10⁻¹-2×10⁻² Torr) to give 39.25 g (89%) of 2-cyanoacetic acid diethylamide in the main fraction.

### EXAMPLE 3

### Preparation of Entacapone in the Polymorphous Form D

### 3.1. Knoevenagel Condensation and Subsequent Isomerization

A mixture of 120 g of 3,4-dihydroxy-5-nitrobenzaldehyde, 94.56 g of N,N-diethyl-2-cyanoacetamide, 3.76 g of acetic acid and 4.58 g of diethylamine in 432 g of toluene was heated in a water separator. When the reaction was almost complete (E/Z isomer ratio=70/30), 109 g of acetonitrile and 38.4 g of active charcoal were added and the mixture was refluxed for 0.5-4 h. While still hot, the suspension was filtered on 14.0 g of Célite and the solid was then rinsed with 66.1 g of acetonitrile. The solution was placed in a receiver with 432 g of toluene. The temperature was not supposed to exceed 20° C. When the addition was complete, 43.2 g of a 33% solution of HBr in acetic acid were added and the mixture was stirred overnight at room temperature. The suspension was then cooled to 0-5° C. and filtered with suction. The moist crude product was elutriated in a mixture of 67.2 g of isopropanol and 100.8 g of water and washed with 200 g of water. The yield was 143.21 g (73.8%, corrected for content).

### 3.2. Preparation of Entacapone in the Polymorphous Form D

Instruction 1: 131.72 g of the product obtained under 3.1. were dissolved in 381.8 g of acetone and 38.2 g of water, with heating to 58° C. While still hot, this solution was added to a cold (0° C.) mixture of 1211.2 g of water, 37.5 g of acetone and 0.3 g of entacapone (polymorphous form D) in such a way that the internal temperature was maintained at 0-12° C. The suspension was then filtered with suction and the product was elutriated in 1300 g of water for 5-10 min at 0° C. After a repeat filtration, the product was washed with 130 g of water. The yield was 126.45 g (95.9%, corrected for content).

Instruction 2: 5.00 g of the product obtained under 3.1. were dissolved in 14.3 g of THF in a round-bottom flask at the boiling point. While still hot at a temperature just below the boiling point, this solution was poured into 124 g of ice-water, the flask was then rinsed with 4.0 g of THF and this solution was also added to the ice-water. The suspension was filtered at an internal temperature of 10° C. and the filter cake was washed with 15 g of ice-water and dried for 15 h at 50° C. The yield was 4.93 g (97.7%, corrected for content).

Instruction 3: 5.00 g of the product obtained under 3.1. were dissolved in 12.0 g of n-propanol at the boiling point. While still hot at a temperature just below the boiling point, this solution was poured into 40.0 g of ice-water. The suspension was filtered at an internal temperature of 23° C. and the filter cake was washed with 10 g of ice-water and dried for 15 h at 70° C. The yield was 4.64 g (93.1%, corrected for content).

### EXAMPLE 4

### Preparation of Entacapone in the Polymorphous Form C

5.00 g of entacapone were dissolved in 189.3 g of toluene, with heating, and added at 95° C. to 267 g of hot n-heptane, causing immediate crystallization. Half of the suspension was filtered hot at 90° C. to give 2.23 g of entacapone in the polymorphous form C. The second half of the suspension was cooled to room temperature and filtered after 14 h to give 2.57 g of entacapone, likewise in the polymorphous form C.

### EXAMPLE 5

### Preparation of Entacapone in the Polymorphous Form E

Instruction 1: 10.00 g of entacapone were dissolved in 28.0 g of THF, with heating, and, while still hot, added to 120 g of cold n-hexane so that the internal temperature did not exceed 10° C., causing immediate crystallization. The suspension was filtered and dried for 15 h at 50° C. to give 9.69 g of entacapone in the polymorphous form E.

Instruction 2: 10.00 g of entacapone were dissolved in 28.0 g of THF, with heating, and, while still hot, added to 120 g of cold n-pentane so that the internal temperature did not exceed 10° C., causing immediate crystallization. The suspension was filtered and dried for 15 h at 50° C. to give 9.72 g of entacapone in the polymorphous form E.

Instruction 3: 10.00 g of entacapone were dissolved in 28.0 g of THF, with heating, and, while still hot, added to 120 g of cold cyclohexane so that the internal temperature did not exceed 10° C., causing immediate crystallization. The suspension was filtered and dried for 15 h at 50° C. to give 9.56 g of entacapone in the polymorphous form E.

Instruction 4: 5.00 g of entacapone were dissolved in 41.4 g of isopropanol at the boiling point. This solution was cooled to 68° C. and poured into 126.0 g of n-hexane (internal temperature: 68° C.). The suspension was immediately filtered and the filter cake was dried for 20 h at 50° C. The yield was 4.08 g (83.2%, corrected for content).

1. A crystalline form C of entacapone, characterized by the following XRD data:

| | Lattice spacing | Rel. intensity |
|---|---|---|
| Angle 2 theta (°) | d (Å) | I/Imax (%) |
| 5.61 | 15.77 | 100 |
| 11.43 | 7.78 | 1 |
| 14.75 | 6.06 | 2 |
| 17.23 | 5.21 | 5 |
| 18.81 | 4.78 | 2 |
| 20.89 | 4.32 | 1 |
| 23.13 | 3.92 | 17 |
| 25.23 | 3.62 | 2 |
| 26.87 | 3.41 | 3 |
| 29.03 | 3.18 | 1 |
| 32.17 | 2.90 | 2 |

2. A crystalline form D of entacapone, characterized by the following XRD data:

| | Lattice spacing | Rel. intensity |
|---|---|---|
| Angle 2 theta (°) | d (Å) | I/Imax (%) |
| 6.84 | 12.95 | 99 |
| 11.84 | 7.51 | 6 |
| 12.12 | 7.34 | 7 |
| 13.52 | 6.59 | 49 |
| 14.8 | 6.04 | 23 |
| 15.56 | 5.75 | 40 |
| 16.54 | 5.42 | 31 |
| 16.9 | 5.30 | 22 |
| 17.98 | 4.99 | 37 |
| 18.84 | 4.77 | 12 |
| 19.06 | 4.72 | 13 |
| 20.72 | 4.36 | 18 |
| 21.44 | 4.22 | 28 |
| 22.24 | 4.07 | 12 |
| 23.4 | 3.88 | 22 |
| 24 | 3.79 | 39 |
| 24.62 | 3.70 | 76 |
| 25.34 | 3.60 | 51 |
| 26.5 | 3.46 | 65 |
| 27.44 | 3.35 | 100 |
| 28.08 | 3.28 | 51 |
| 29.24 | 3.16 | 15 |
| 29.98 | 3.09 | 17 |

3. A crystalline form E of entacapone, characterized by the following XRD data:

| | Lattice spacing | Rel. intensity |
|---|---|---|
| Angle 2 theta (°) | d (Å) | I/Imax (%) |
| 6.62 | 13.35 | 100 |
| 8.87 | 9.97 | 4 |
| 12.36 | 7.16 | 8 |
| 12.90 | 6.86 | 12 |
| 13.38 | 6.62 | 11 |
| 14.40 | 6.15 | 5 |
| 15.52 | 5.71 | 49 |
| 17.92 | 4.95 | 33 |
| 18.25 | 4.86 | 22 |
| 19.20 | 4.62 | 6 |
| 20.48 | 4.24 | 26 |
| 21.10 | 4.21 | 7 |
| 21.85 | 4.07 | 6 |
| 22.45 | 3.96 | 6 |
| 22.90 | 3.88 | 7 |
| 24.00 | 3.71 | 30 |
| 24.64 | 3.61 | 36 |
| 25.85 | 3.45 | 77 |
| 27.32 | 3.26 | 20 |

4. A process for the preparation of the crystalline form C of entacapone as claimed in claim 1, characterized in that entacapone is crystallized from a mixture of at least one aromatic and at least one aliphatic hydrocarbon.

5. The process as claimed in claim 4, characterized in that the aromatic hydrocarbon used is toluene and the aliphatic hydrocarbon used is n-heptane.

6. A process for the preparation of the crystalline form D of entacapone as claimed in claim 2, characterized in that
a) entacapone is dissolved in a water-miscible solvent and this solution is added to water or a mixed aqueous system; or
b) entacapone is crystallized from a non-acidic solvent or a solvent mixture with at least one non-acidic component, in the presence of a strong acid.

7. The process as claimed in claim 6, variant a), characterized in that it is carried out in THF/water, acetone/water, acetone/DMSO/water or n-propanol/water.

8. The process as claimed in claim 6, variant b), characterized in that it is carried out in toluene/acetonitrile or toluene/acetonitrile/acetic acid.

9. The process as claimed in claim 6, variant b), or claim 8, characterized in that the acid used is hydrogen bromide.

10. A process for the preparation of the crystalline form E of entacapone as claimed in claim 3, characterized in that entacapone is dissolved in a polar aprotic or alcoholic solvent and this solution is added to an aliphatic hydrocarbon immiscible with this solvent, in which entacapone is insoluble.

11. The process as claimed in claim 10, characterized in that it is carried out in THF/n-hexane, THF/n-pentane, THF/cyclohexane or isopropanol/n-hexane.

12. The process as claimed in claim 6, characterized in that crude entacapone is used.

13. The process as claimed in claim 8, characterized in that entacapone is used in situ in the form of the product of a Knoevenagel condensation of 3,4-dihydroxy-5-nitrobenzaldehyde and 2-cyanoacetic acid diethylamide.

14. The process as claimed in claim 8, characterized in that the acid used is hydrogen bromide and the process is carried out in toluene/acetonitrile/acetic acid.

15. The crystalline form C, D or E of entacapone as claimed in claim 1, for use as a therapeutic active ingredient.

16. A drug containing the crystalline form C, D or E of entacapone as claimed in claim 1, and a therapeutically inert excipient.

17. The drug as claimed in claim 16 additionally containing levodopa and a decarboxylase inhibitor.

18. The use of the crystalline form C, D or E of entacapone as claimed in claim 1, optionally in combination with levodopa and a decarboxylase inhibitor, for the treatment of Parkinson's disease or for the preparation of corresponding drugs.

19. A process for the preparation of entacapone by a Knoevenagel condensation of 3,4-dihydroxy-5-nitrobenzaldehyde and N,N-diethyl-2-cyanoacetamide, characterized in that the catalyst used for this condensation is diethylamine/acetic acid.

20. The process as claimed in claim 19, characterized in that the N,N-diethyl-2-cyanoacetamide used has been prepared by reacting cyanoacetic acid with diethylamine in the presence of dicyclohexylcarbodiimide.

21. The process as claimed in claim 19, characterized in that the 3,4-dihydroxy-5-nitrobenzaldehyde used has been prepared by the demethylation of 5-nitrovanillin with AlCl₃/pyridine in chlorobenzene.
